(19) United States
(12) Patent Application Publication
(10) Pub. No.: US 2017/0029870 A1
(43) Pub. Date: Feb. 2, 2017

(54) LYOPHILIZED REAGENT, MIXED REAGENT SOLUTION, AND METHOD FOR STORING LYOPHILIZED REAGENT

(71) Applicant: Seiko Epson Corporation, Tokyo (JP)

(72) Inventor: Kotaro IDEGAMI, Chino (JP)

(21) Appl. No.: 15/219,579

(22) Filed: Jul. 26, 2016

(30) Foreign Application Priority Data
Jul. 29, 2015 (JP) 2015-149483

(51) Int. Cl. C12Q 1/68; C12N 9/12; C12N 9/96

(52) U.S. Cl. CPC C12Q 1/686; C12N 9/96; C12N 9/1276; C12Y 207/07049; C12Y 207/07; C12N 9/1241

(57) ABSTRACT

A lyophilized reagent according to the invention is a lyophilized reagent for a nucleic acid amplification reaction obtained by lyophilizing a reaction mixture containing a reagent to be used for a nucleic acid amplification reaction and a sugar, wherein the sugar is contained in an amount of 72 mass % or more and 86 mass % or less with respect to the concentration of the solids contained in the lyophilized reagent.

### BACKGROUND

1. Technical Field

The present invention relates to a lyophilized reagent, a mixed reagent solution to be used for preparing this lyophilized reagent, and a method for storing a lyophilized reagent.

2. Related Art

In the field of biochemistry, a PCR (Polymerase Chain Reaction) technique has been established. Recently, amplification accuracy and detection sensitivity of a PCR method have been improved, and it has become possible to amplify an extremely small amount of a sample (DNA or the like) and to perform detection and analysis of the sample. The PCR is a method for amplifying a target nucleic acid by subjecting a solution (reaction solution) containing a nucleic acid (target nucleic acid) to be amplified and a reagent to thermal cycling. For the thermal cycling in PCR, a method for performing thermal cycling at temperatures in two stages or three stages is generally used.

It is generally very difficult to store a nucleic acid amplification reaction reagent to be used in PCR for a long period of time because an enzyme to be used in the reagent shows low storage performance. In particular, a reverse transcriptase cannot be kept for even 1 day in a state of an aqueous solution even if the aqueous solution is stored at 4° C., and the activity is decreased. Therefore, it has been known that the storage stability of the reagent is improved by adding an additive to the reagent and lyophilizing the reagent (see, for example, JP-T-2000-514298 (PTL 1)). Here, by including trehalose at a concentration of 8 to 12% (weight/volume) with respect to the reaction mixture, the storage stability of the lyophilized reaction mixture is improved.

However, in the case where trehalose is contained at a concentration of 8 to 12% (weight/volume) with respect to the reaction mixture, the react ion mixture lyophili zed under vacuum maintains its activity for 6 months in a temperature range from 0° C. to 10° C., but is completely inactivated within 1 month at a temperature of 15° C. or higher and 0° C. or lower, and cannot respond to the need of longer-term storage stability.

On the other hand, in order to obtain long-term storage stability by maintaining the lyophilized state of a reagent, a vial bottle containing a lyophilized reagent is stored in a refrigerator, or in order to avoid the contact between a lyophilized reagent and a water molecule, a vial bottle containing a lyophilized reagent is purged with nitrogen, or a desiccant is packed along with the lyophilized reagent, however, a water molecule is still absorbed in the lyophilized reagent in some cases. Then, the water molecule once absorbed in the lyophilized reagent is never released therefrom, and therefore, even in the case of a lyophilized reagent, long-term storage stability cannot be obtained.

### SUMMARY

An advantage of some aspects of the invention is to provide a lyophilized reagent which has excellent storage stability and also is less likely to cause reaction inhibition in a nucleic acid amplification reaction, a mixed reagent solution to be used for preparing this lyophilized reagent, and a method for storing a lyophilized reagent by solving at least part of the problems described above.

The invention can be implemented as the following aspects or application examples.

### Application Example 1

This application example of the invention is directed to a lyophilized reagent for a nucleic acid amplification reaction obtained by lyophilizing a reaction mixture containing a reagent to be used for a nucleic acid amplification reaction and a sugar, wherein the sugar is contained in an amount of 72 mass % or more and 86 mass % or less with respect to the total mass of the solids contained in the lyophilized reagent.

According to this application example, a lyophilized reagent which has excellent storage stability and also is less likely to cause reaction inhibition in a nucleic acid amplification reaction can be provided by adjusting the concentration of the sugar contained in the lyophilized reagent.

### Application Example 2

In the lyophilized reagent according to the application example, the sugar may be a disaccharide or a trisaccharide.

### Application Example 3

In the lyophilized reagent according to the application example, the lyophilized reagent may be stored in a hydrophobic liquid.

### Application Example 4

In the lyophilized reagent according to the application example, the hydrophobic liquid may contain at least one member selected from a silicone oil, a mineral oil, and paraffin.

### Application Example 5

In the lyophilized reagent according to the application example, the sugar may contain trehalose.

### Application Example 6

In the lyophilized reagent according to the application example, the reagent may contain a reverse transcriptase.

### Application Example 7

In the lyophilized reagent according to the application example, the reagent may contain a polymerase and dNTP.

### Application Example 8

In the lyophilized reagent according to the application example, the reagent may contain at least one member selected from a primer and a probe.

### Application Example 9

In the lyophilized reagent according to the application example, the sugar may be contained in an amount of 77 mass % or more and 84 mass % or less with respect to the total mass of the solids contained in the lyophilized reagent.

### Application Example 10

This application example of the invention is directed to a mixed reagent solution for a nucleic acid amplification reaction including a reaction mixture containing a reagent to be used for a nucleic acid amplification reaction and a sugar, wherein the concentration of the sugar in the mixed reagent solution is 10% (mass/volume) or more and 24% (mass/volume) or less.

By adjusting the concentration of the sugar contained in the mixed reagent solution according to the above application example, a lyophilized reagent obtained by lyophilizing this mixed reagent solution has excellent storage stability and also is less likely to cause reaction inhibition in a nucleic acid amplification reaction.

### Application Example 11

This application example of the invention is directed to a method for storing a lyophilized reagent including storing the lyophilized reagent according to the application example in a hydrophobic liquid.

According to this application example, by storing a lyophilized reagent in which the concentration of the sugar contained therein is adjusted in a hydrophobic liquid, the lyophilized reagent can be prevented from coming into contact with a water molecule, and thus, the storage stability of the lyophilized reagent can be improved.

### BRIEF DESCRIPTION OF THE DRAWINGS

The invention will be described with reference to the accompanying drawings, wherein like numbers reference like elements.

FIG. 1 is a graph showing the results of performing real-time RT-PCR under standard conditions with respect to the trehalose concentration dependency.

FIG. 2 is a graph with respect to the storage stability after 1 week by the addition of trehalose.

FIG. 3 is a graph with respect to the storage stability after 1 month by the addition of trehalose.

FIG. 4 is a graph with respect to the storage stability by the addition of trehalose and an oil.

FIG. 5 is a graph with respect to the storage stability by the addition of trehalose.

FIG. 6 is a graph with respect to the storage stability by the addition of sucrose.

FIG. 7 is a graph with respect to the storage stability by the addition of raffinose.

FIG. 8 is a graph with respect to the storage stability by the addition of polypropylene.

### DESCRIPTION OF EXEMPLARY EMBODIMENTS

Hereinafter, preferred embodiments of the invention will be described. The below-mentioned embodiments describe an example of the invention. Further, the invention is not limited to the following embodiments and also includes various modifications made within the scope not changing the gist of the invention.

### 1. Lyophilized Reagent

The lyophilized reagent according to this embodiment is a lyophilized reagent for a nucleic acid amplification reaction obtained by lyophilizing a reaction mixture containing a reagent to be used for a nucleic acid amplification reaction and a sugar, wherein the sugar is contained in an amount of 72 mass % or more and 86 mass % or less with respect to the total mass of the solids contained in the lyophilized reagent. Hereinafter, the respective components contained in the lyophilized reagent according to this embodiment and the like will be described.

### 1.1. Reaction Mixture

The lyophilized reagent according to this embodiment includes a reaction mixture containing a reagent to be used for a nucleic acid amplification reaction. The reaction mixture is a material obtained by lyophilizing an aqueous solution in which a reagent to be used for a nucleic acid amplification reaction is dissolved in, for example, sterile water, purified water such as distilled water or ion exchanged water, or such water.

Examples of the reagent to be used for a nucleic acid amplification reaction include a polymerase and dNTP, however, it is preferred to contain a primer for a nucleic acid amplification reaction and/or a probe for a nucleic acid amplification reaction, or a buffer. The lyophilized nucleic acid amplification reaction reagent may contain a reverse transcriptase. In such a case, a primer for reverse transcription may be contained aside from the primer for a nucleic acid amplification reaction, or may be shared with the primer for a nucleic acid amplification reaction.

The polymerase is not particularly limited, however, examples thereof include a DNA polymerase. As the DNA polymerase, a heat resistant enzyme or an enzyme for PCR is preferred, and there are a lot of commercially available products, for example, Taq polymerase, Tfi polymerase, Tth polymerase, modified forms thereof, and the like, however, a DNA polymerase capable of performing hot start is preferred.

dNTP represents a mixture of four types of deoxyribonucleotide triphosphates. That is, dNTP represents a mixture of dATP (deoxyadenosine triphosphate), dCTP (deoxycytidine triphosphate), dGTP (deoxyguanosine triphosphate), and dTTP (thymidine triphosphate).

Also the reverse transcriptase is not particularly limited, and for example, a reverse transcriptase derived from avian myeloblast virus, Ras-associated virus type 2, mouse Moloney murine leukemia virus, or human immunodefficiency virus type 1, or the like can be used, however, a heat resistant enzyme is preferred. There are also a lot of commercially available products of the heat resistant enzyme, and therefore, such a product can be used.

Here, in the case where the reagent to be used in the reaction mixture is dissolved in water to prepare a reaction solution, the concentrations of the polymerase, dNTP, probe, primer, buffer, and salt are appropriately set according to the reaction to be used, however, for example, the concentration of dNTP may be set to 10 to 1000 mM, preferably 100 to 500 mM, the concentration of Mg²⁺ may be set to 1 to 100 mM, preferably 5 to 10 mM, and the concentration of Cl⁻ may be set to 1 to 2000 mM, preferably 200 to 700 mM. The total ion concentration is not particularly limited, but may be higher than 50 mM, preferably higher than 100 mM, more preferably higher than 120 mM, further more preferably higher than 150 mM, still further more preferably higher than 200 mM. The upper limit thereof is preferably 500 mM or less, more preferably 300 mM or less, further more preferably 200 mM or less. The oligonucleotides for primers are used at 0.1 to 20 mM, respectively.

### 1.2. Sugar

The lyophilized reagent according to this embodiment contains a sugar, and the sugar is contained in an amount of 72 mass % or more and 86 mass % or less with respect to the total mass of the solids contained in the lyophilized reagent. The enzyme or the like to be used in the reagent has poor storage stability, and therefore, in order to maintain the activity thereof for a long period of time, the sugar is added in a predetermined amount, and further, a mixed reagent solution containing the sugar and the reaction mixture is lyophilized.

The sugar contained in the lyophilized reagent is not particularly limited as long as it is a sugar which has a function of maintaining a nucleic acid amplification reaction without inactivating the lyophilized enzyme or the like when the reagent to be used for a nucleic acid amplification reaction is lyophilized, that is, a sugar which has excellent storage stability and functions as a cryoprotective agent, but is preferably a sugar which has high affinity for water and does not react with other substances such as an enzyme. Examples of the sugar include sucrose, trehalose, raffinose, and melezitose, which are nonreducing sugars, among disaccharides and trisaccharides.

Among disaccharides and trisaccharides, it is particularly preferred to use trehalose because the function as a cryoprotective agent is high. It is presumed that trehalose prevents the lyophilized reagent from coming into contact with a water molecule and improves the storage stability of the lyophilized reagent because of its strong hydration force.

In this embodiment, the content of the sugar is 72 mass % or more and 86 mass % or less, preferably 77 mass % or more and 84 mass % or less with respect to the total mass (100 mass %) of the solids contained in the lyophilized reagent. When the content of the sugar is 72 mass % or more and 86 mass % or less, the storage stability of the lyophilized reagent is favorable, and the reaction inhibition is less likely to occur when the lyophilized reagent is redissolved and a nucleic acid amplification reaction is performed.

In particular, since the lyophilized reagent according to this embodiment contains the sugar in an amount of 72 mass % or more and 86 mass % or less with respect to the total mass (100 mass %) of the solids contained in the lyophilized reagent, which is larger than that in the related art, so as to occupy the majority of the lyophilized material, it becomes possible to maintain the activity comparable to that before lyophilization over a long period of time even at room temperature.

The concentration of the sugar contained in the lyophilized reagent is 10% (mass/volume) or more and 24% (mass/volume) or less, preferably 13% (mass/volume) or more and 21% (mass/volume) or less when it is in a state of a mixed reagent solution with the reaction mixture before lyophilization. In the case where the concentration of the sugar contained in the lyophilized reagent in a state of a mixed reagent solution before lyophilization is 10% (mass/volume) or more and 24% (mass/volume) or less, the reaction inhibition by the added sugar does not occur, and also the storage stability is excellent and the reagent after lyophilization can be stored for a long period of time while maintaining the activity.

### 1.3. Method for Preparing Lyophilized Reagent

The lyophilized reagent according to this embodiment can be prepared by lyophilizing a mixed reagent solution including a reaction mixture containing a reagent to be used for a nucleic acid amplification reaction and a predetermined amount of a sugar. For example, in a container for a nucleic acid amplification reaction, a mixed reagent solution containing a buffer for a nucleic acid amplification reaction, a reaction mixture in an amount for one reaction, and a sugar is placed and lyophilized by being left at low temperature and low pressure for a predetermined time, whereby the reaction mixture and the sugar are solidified and adhered to the bottom of the container. At this time, as the amount of the buffer for a nucleic acid amplification reaction is smaller, the reagent is solidified and adhered to the bottom of the container such that the reagent is small and hard.

The lyophilization temperature is not particularly limited, but is preferably set to a temperature lower than the collapse temperature of the reagent to be lyophilized, and is preferably −80° C. or higher. The average opening diameter of micropores becomes smaller when the mixed reagent solution is rapidly frozen, and the resolubility and storage stability become favorable when the average opening diameter of micropores is smaller. Therefore, it is preferred to lyophilize the mixed reagent solution after undergoing a step of keeping the mixed reagent solution in a liquid at a temperature lower than the lyophilization temperature by a method of keeping the mixed reagent solution in liquid nitrogen or the like as preliminary freezing before lyophilization, followed by vaporization of the liquid used.

The pressure at the time of low pressure is not particularly limited, but is preferably 100 mmHg or less, and most preferably 20 mmHg or less. The time for keeping the pressure low is also not particularly limited, but is preferably from 2 to 24 hours, and most preferably about 8 hours.

By lyophilizing the mixed reagent solution in a container to be used for the container for a nucleic acid amplification reaction in this manner, the reaction mixture and the sugar are solidified and adhered to the bottom of the container. Thereafter, in the case where a hydrophobic liquid is added as described below, it is preferred to add the hydrophobic liquid so as to fill the container. In the case where a wax is used as the hydrophobic liquid, the wax is added in a molten state. In the case where an oil is used, it is preferred to dehydrate the oil with silica gel and/or a molecular sieve, or the like beforehand, and it is preferred to perform the addition of the oil in a glovebox or the like in which the water content is controlled to a sufficiently low level. Further, it is preferred that after the oil is added, the container is lightly centrifuged so as to remove air bubbles.

### 1.4. Physical Properties

The lyophilized reagent according to this embodiment is preferably a porous material which is in the form of a sponge or a cake and has a lot of micropores (air holes) in which air bubbles are trapped, and according to this, the resolubility when the lyophilized reagent is redissolved and used after lyophilization is improved. The average opening diameter of the micropores becomes smaller when the mixed reagent solution is rapidly frozen, and the solubility and storage stability become favorable when the average opening diameter of the micropores is smaller. The average opening diameter of the pores (which is determined by, for example, measuring the diameters of the pores in the cross section a fixed number of times under a microscope, and averaging the diameters) is preferably 20 μm or less, more preferably 10 μm or less, further more preferably 5 μm or less.

### 1.5. Method for Storing Lyophilized Reagent

The lyophilized reagent according to this embodiment prepared by the above-mentioned preparation method is solidified and adhered to the bottom of the container along with the sugar in the container to be used for the container for a nucleic acid amplification reaction. The lyophilized reagent according to this embodiment has storage stability improved by containing the sugar in a large amount, and can withstand long-term storage at room temperature even in a lyophilized form as it is, however, in order to achieve long-term storage stability by further eliminating the contact with a water molecule, it is preferred to store the lyophilized reagent in a hydrophobic liquid by adding the hydrophobic liquid to the container in which the reaction mixture and the sugar are solidified and adhered to the bottom. By doing this, it becomes more difficult for the lyophilized reagent to be exposed to a water molecule, and thus, the storage stability performance can be further improved.

Examples of the hydrophobic liquid include an oil and a wax. These liquids can be used alone, however, for example, by adding a molten wax to the lyophilized reagent obtained by lyophilization before adding an oil, and then, by adding an oil thereto after the wax is solidified, the lyophilized reagent can be prevented from diffusing in the oil.

Here, the wax refers to an organic material which is a liquid or a solid at room temperature, and in the case where the wax is a solid at room temperature, the solid is converted to a liquid by heating. The wax which can be used in the invention has a melting point of 31° C. or higher, preferably 36° C. or higher, more preferably 41° C. or higher, further more preferably 46° C. or higher, and is 100° C. or lower, preferably 90° C. or lower, more preferably 80° C. or lower, further more preferably 70° C. or lower, and is preferably composed of a neutral fat, a higher fatty acid, a hydrocarbon, or the like. The wax is not particularly limited, and for example, a petroleum-derived wax such as paraffin wax or microcrystalline wax, an animal-derived wax such as beeswax, wool wax, or spermaceti, or a vegetable-derived wax such as carnauba wax, rosin, candelilla wax, or Japan wax can be used. Other than these, El Christa (registered trademark, Idemitsu Kosan Co., Ltd.), Nissan Elector (registered trademark, NOF Corporation), Poem (registered trademark, Riken Vitamin Co., Ltd.), Rikemal (registered trademark, Riken Vitamin Co., Ltd.), Neo Wax (registered trademark, Yasuhara Chemical Co., Ltd.), Hi-Wax (registered trademark, Mitsui Chemicals, Inc.), Silicone Wax (trademark, Dow Corning Toray Co. Ltd.), or the like can be used.

The type of the oil is not particularly limited, and a mineral oil, a silicone oil (such as 2CS silicone oil), a vegetable oil, or the like can be used.

Among these, as the hydrophobic liquid, at least one member selected from a silicone oil, a mineral oil, and paraffin can be contained.

It is preferred that the hydrophobic liquid is dehydrated beforehand. By dehydrating the hydrophobic liquid beforehand, it becomes more difficult for the lyophilized reagent to be exposed to a water molecule, and thus, the storage stability performance can be improved.

### 1.6. Method for Redissolving Lyophilized Reagent and Usage Thereof

In the case where the container used can be used in a nucleic acid amplification device such as a PCR device, the container for a nucleic acid amplification reaction containing this reagent for a nucleic acid amplification reaction can be used for a nucleic acid amplification reaction as it is. Specifically, an appropriate amount of pure water or a buffer containing a DNA to be amplified is added so as to soak the lyophilized reagent, and a nucleic acid amplification reaction may be started. The amount of pure water or the buffer can be easily determined so that the final concentrations of the salts and the like are suitable for the nucleic acid amplification reaction. Incidentally, the reagent for a nucleic acid amplification reaction may be sufficiently dissolved by performing a heating treatment or a shaking treatment before the reaction.

In the case where the lyophilized nucleic acid amplification reaction reagent contains a reverse transcriptase, an appropriate amount of pure water or a buffer containing an RNA may be added so as to soak the lyophilized reagent, and in such a case, a reverse transcription reaction can be performed, and thereafter, a nucleic acid amplification reaction can be performed.

Also in the case where a hydrophobic liquid is contained, the method for dissolving the lyophilized reagent and the usage thereof are the same. However, in the case where a wax is used as the hydrophobic liquid and the solid wax covers the lyophilized nucleic acid amplification reaction reagent, the lyophilized reagent is heated to a temperature at which the wax is melted, and after the wax is melted, an appropriate amount of pure water or a buffer containing a nucleic acid may be added as described above. In this case, even in the case where the lyophilized reagent is stored in the hydrophobic liquid, the reaction occurs without being inhibited by the hydrophobic liquid.

In the case where the above-mentioned pure water or buffer is added so that the volume of a solution obtained by redissolving the lyophilized reagent is the same as the volume of the mixed reagent solution before lyophilization, the concentration of the sugar contained in the solution is 10% (mass/volume) or more and 24% (mass/volume) or less in the same manner as that in the mixed reagent solution before lyophilization. When the concentration of the sugar contained in the solution after redissolution is 10% (mass/volume) or more and 24% (mass/volume) or less, reaction inhibition by the sugar added does not occur in the nucleic acid amplification reaction.

The lyophilized reagent according to this embodiment can withstand long-term storage at room temperature even in a lyophilized form as it is because of including the sugar in a predetermined amount, and therefore, in the case where a nucleic acid amplification reaction is performed by adding an appropriate amount of pure water or a buffer containing a DNA to be amplified after storage, the activity comparable to that before lyophilization is maintained without causing reaction inhibition. The lyophilized reagent according to this embodiment realizes, for example, storage performance for nearly 2 years at a temperature of 20° C. by a combination with a hydrophobic liquid.

### 2. Examples

Hereinafter, the invention will be more specifically described by way of Examples, however, the invention is not limited only to these Examples.

### 2.1. Study of Reaction Inhibition Depending on Concentration of Trehalose

First, the effect of trehalose added to a nucleic acid amplification reaction reagent on the amplification reaction of InfA was studied.

1.6 μL of a mixed reagent solution having the following composition and obtained by adding trehalose to a nucleic acid amplification reaction reagent was introduced into a 200-μL PCR tube (a container for a nucleic acid amplification reaction) manufactured by Eppendorf AG. Each mixed reagent solution was prepared by adding 2 μL of an aqueous solution of trehalose adjusted at a concentration ranging from 0 to 800 mM to the nucleic acid amplification reaction reagent, and thereafter adding distilled water thereto up to 10 μL. That is, the concentration of trehalose in the mixed reagent solution before lyophilization was from 0 to 27.1% (mass/volume), and the concentration of trehalose in the lyophilized reagent after lyophilization was from 0 to 87 mass %. Thereafter, in order to lyophilize the solution, the solution was frozen in a deep freezer at −80° C., and the sufficiently frozen sample was quickly placed in a lyophilizer. Subsequently, lyophilization was performed overnight at room temperature in a reduced pressure environment at 10 Pa. Subsequently, 1.6 μL of an aqueous solution containing a template of influenza virus A was added dropwise into the PCR tube containing the lyophilized material of the mixed reagent, thereby redissolving the lyophilized material, and by using the resulting solution, real-time RT-PCR was performed according to the following protocol.

### Composition of Mixed Reagent Solution

| | |
|---|---|
| SuperScript III (reverse transcriptase) | 0.2 μL |
| Buffer | 2 μL |
| 10 mM dNTP | 0.25 μL |
| 20 mM forward primer | 0.8 μL |
| 20 mM reverse primer | 0.8 μL |
| 10 mM probe | 0.5 μL |
| 0 to 800 mM aqueous solution of | 2 μL |
| trehalose | |
| InfA | 0.5 μL |
| D.W. | up to |
| | 10 μL |

Primer
Primer F: GAC CAA TCC TGT CAC CTC TGA C
Primer R: AGG GCA TTT TGG ACA AAG CGT CTA
Probe:
TaqMan probe: FAM- TGC AGT CCT CGC TCA CTG GGC
ACG -TAMRA

The conditions for the RT-PCR are as follows.

Reverse transcription: 50° C., 60 sec

Amplification reaction

Denaturation: 105° C., 5 sec

Annealing/elongation: 57° C., 20 sec

Denaturation and Annealing/elongation: 50 cycles

Liquid droplet size: 1.6 μL

Experimental device: rise and fall system

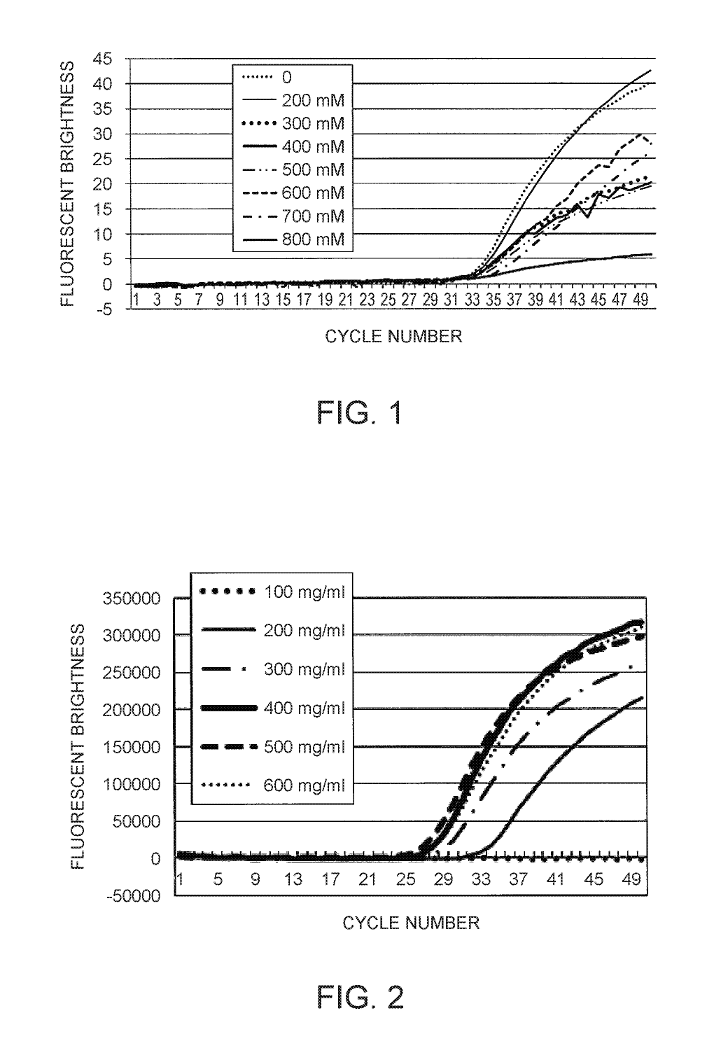

As shown in FIG. 1, in the case where the concentration of the aqueous solution of trehalose added was up to 700 mM (the concentration of trehalose in the mixed reagent solution before lyophilization was 23.8% (mass/volume)), an effect on the Ct value was not so much observed, however, in the case where the concentration of trehalose added was 800 mM (the concentration of trehalose in the mixed reagent solution before lyophilization was 27.1% (mass/volume)), a significant delay in the Ct value was confirmed, and it was found that the amplification reaction was inhibited. From these results, it was found that the concentration of trehalose contained in the mixed reagent in a state of a solution before lyophilization is preferably 24% (mass/volume) (the concentration of trehalose in the lyophilized material is 86 mass %) or less.

### 2.2. Study of Storage Stability Effect of Trehalose

Next, the storage stability effect of trehalose contained in the lyophilized reagent was studied.

1.6 μL of a mixed reagent solution in which trehalose was added to a nucleic acid amplification reaction reagent was introduced into a 200-μL PCR tube (a container for a nucleic acid amplification reaction) manufactured by Eppendorf AG. Thereafter, in order to lyophilize the solution, the solution was frozen in a deep freezer at −80° C., and the sufficiently frozen sample was quickly placed in a lyophilizer. Subsequently, lyophilization was performed overnight at room temperature in a reduced pressure environment at 10 Pa. Immediately after lyophilization, the lyophilized material was stored in a thermostatic bath at 30° C. After 2 days, 1 week, and 1 month, 1.6 μL of an aqueous solution containing a template of influenza virus A was added dropwise into the PCR tube containing the lyophilized material, thereby redissolving the lyophilized material, and by using the resulting solution, real-time RT-PCR was performed under the same conditions as in 2.1.

The composition of the mixed reagent solution is the same as in the above 2.1. except that an aqueous solution of trehalose at a concentration of 100 to 600 mg/mL was used as the aqueous solution of trehalose to be added in an amount of 2 μL to the mixed reagent solution. That is, the concentration of trehalose in the mixed reagent solution before lyophilization was from 1.9 to 13.9% (mass/volume), and the concentration of trehalose in the lyophilized reagent after lyophilization was from 32 to 78 mass %.

Immediately after lyophilization and after 2 days, an amplification reaction occurred in the same manner at any concentration. On the other hand, as shown in FIG. 2, after 1-week storage, an amplification reaction was not observed and a storage stability effect was not obtained when the concentration of the aqueous solution of trehalose added was 100 mg/mL, that is, when the concentration of trehalose in the mixed reagent solution before lyophilization was 1.9% (mass/volume) and the concentration of trehalose in the lyophilized reagent after lyophilization was 32 mass %. In addition, when the concentration of the aqueous solution of trehalose added was 200 and 300 mg/mL, that is, when the concentration of trehalose in the mixed reagent solution before lyophilization was from 3.9 to 6.1% (mass/volume) and the concentration of trehalose in the lyophilized reagent after lyophilization was from 50 to 61 mass %, a delay in the Ct value was confirmed and the storage stability effect was low. On the other hand, when the concentration of the aqueous solution of trehalose added was from 400 to 600 mg/mL, that is, when the concentration of trehalose in the mixed reagent solution before lyophilization was from 8.5 to 13.9% (mass/volume) and the concentration of trehalose in the lyophilized reagent after lyophilization was from 68 to 78 mass %, a high storage stability effect was obtained and also reaction inhibition was not confirmed.

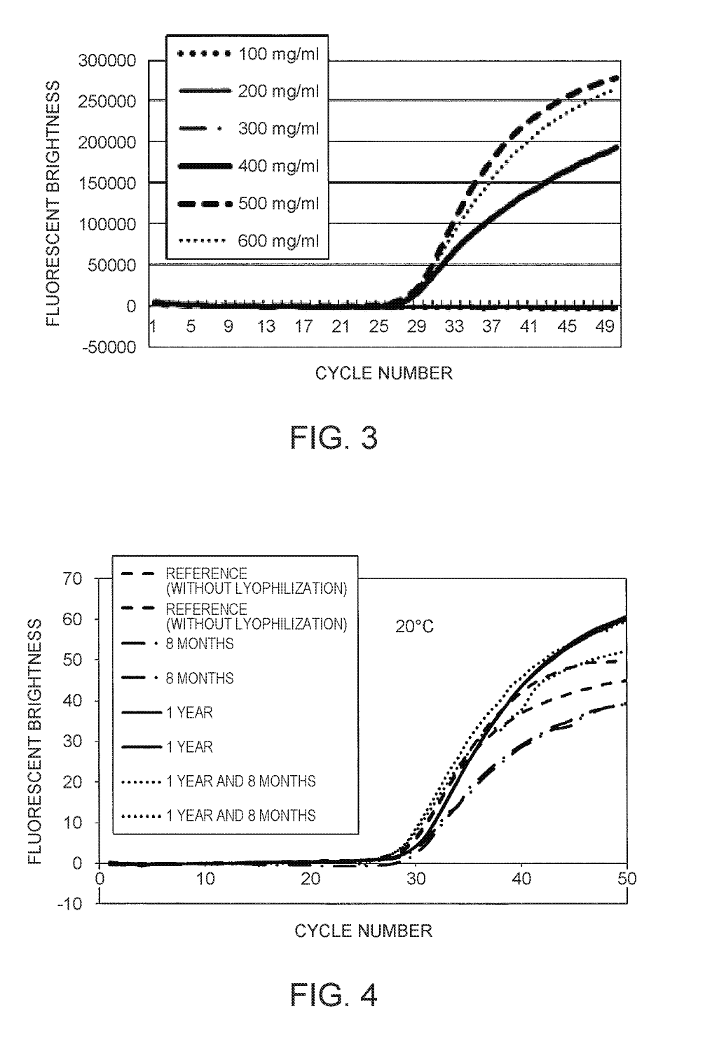

Next, as shown in FIG. 3, after 1-month storage, an amplification reaction was not observed and a storage stability effect was not obtained when the concentration of the aqueous solution of trehalose added was from 100 to 300 mg/mL, that is, when the concentration of trehalose in the mixed reagent solution before lyophilization was from 1.9 to 6.1% (mass/volume) and the concentration of trehalose in the lyophilized reagent after lyophilization was from 32 to 61 mass %. On the other hand, when the concentration of the aqueous solution of trehalose added was from 400 to 600 mg/mL, that is, when the concentration of trehalose in the mixed reagent solution before lyophilization was from 8.5 to 13.9% (mass/volume) and the concentration of trehalose in the lyophilized reagent after lyophilization was from 68 to 78 mass %, a storage stability effect was obtained. In particular, when the concentration of the aqueous solution of trehalose added was from 500 to 600 mg/mL, that is, when the concentration of trehalose in the mixed reagent solution before lyophilization was from 11.1 to 13.9% (mass/volume) and the concentration of trehalose in the lyophilized reagent after lyophilization was from 73.8 to 78 mass %, also reaction inhibition was not observed, and a high storage stability effect was obtained.

From the above results, it was found that when the concentration of the aqueous solution of trehalose added was from 500 to 600 mg/mL, that is, when the concentration of trehalose in the mixed reagent solution before lyophilization was from 11.1 to 13.9% (mass/volume) and the concentration of trehalose in the lyophilized reagent after lyophilization was from 73.8 to 78 mass %, a high storage stability effect was obtained.

### 2.3. Study of Storage Stability by Addition of Trehalose and Oil

Next, the storage stability in the case where a lyophilized reagent in which trehalose was added was stored in an oil was studied.

1.6 μL of a mixed reagent solution in which trehalose was added to a nucleic acid amplification reaction reagent was introduced into a 200-μL PCR tube (a container for a nucleic acid amplification reaction) manufactured by Eppendorf AG. Thereafter, in order to lyophilize the solution, the solution was frozen in a deep freezer at −80° C., and the sufficiently frozen sample was quickly placed in a lyophilizer. Subsequently, lyophilization was performed overnight at room temperature in a reduced pressure environment at 10 Pa. Thereafter, 50 μL of a silicone oil (manufactured by Shin-Etsu Chemical Co., Ltd.) having a viscosity of 2 CS was added dropwise into the tube containing the lyophilized material. The thus obtained lyophilized material was stored at room temperature, and after 8 months, 1 year, and 1 year and 8 months, 1.6 μL of an aqueous solution containing a template of influenza virus A was added dropwise into the PCR tube containing the lyophilized material, thereby redissolving the lyophilized material, and by using the resulting solution, real-time RT-PCR was performed under the same conditions as in 2.1. As comparison, RT-PCR was performed under the same conditions also for 1.6 μL of the solution which was not lyophilized.

The composition of the mixed reagent solution is the same as in the above 2.1. except that the preparation was performed so that the concentration of the aqueous solution of trehalose to be added was 1.6 mmol/mL, that is, the concentration of trehalose in the mixed reagent solution before lyophilization was 18.8% (mass/volume), and the concentration of trehalose in the lyophilized material was 83 mass %.

The results of the experiment performed under the conditions of n=2 are shown in FIG. 4. From FIG. 4, it is found that even after 1 year and 8 months, an amplification reaction was observed in the same manner as in the case of the solution which was not lyophilized, and a possibility that the lyophilized reagent has storage performance for about 2 years at a temperature of 20° C. was suggested by combination of addition of trehalose and the oil.

2.4. Study of Storage Stability Effect of Sugar Other than Trehalose

Finally, the storage stability effect of a sugar other than trehalose was studied.

1.6 μL of a mixed reagent solution in which trehalose was added to a nucleic acid amplification reaction reagent was introduced into a 200-μL PCR tube (a container for a nucleic acid amplification reaction) manufactured by Eppendorf AG. Thereafter, in order to lyophilize the solution, the solution was frozen in a deep freezer at −80° C., and the sufficiently frozen sample was quickly placed in a lyophilizer. Subsequently, lyophilization was performed overnight at room temperature in a reduced pressure environment at 10 Pa. Thereafter, 50 μL of a silicone oil (manufactured by Shin-Etsu Chemical Co., Ltd.) with a viscosity of 2 CS was added dropwise into the tube containing the lyophilized material. Immediately after lyophilization, the lyophilized material was stored in a thermostatic bath at 30° C. After 1 week, and 3 weeks, 1.6 μL of an aqueous solution containing a template of influenza virus A was added dropwise into the PCR tube containing the lyophilized material, thereby redissolving the lyophilized material, and by using the resulting solution, real-time RT-PCR was performed under the same conditions as in 2.1.

The composition of the mixed reagent solution is the same as in the above 2.1. except that as the additive to be added to the mixed reagent solution, trehalose, sucrose, raffinose, and polypropylene were used, and the concentration of each additive in the mixed reagent solution was 10% (mass/volume).

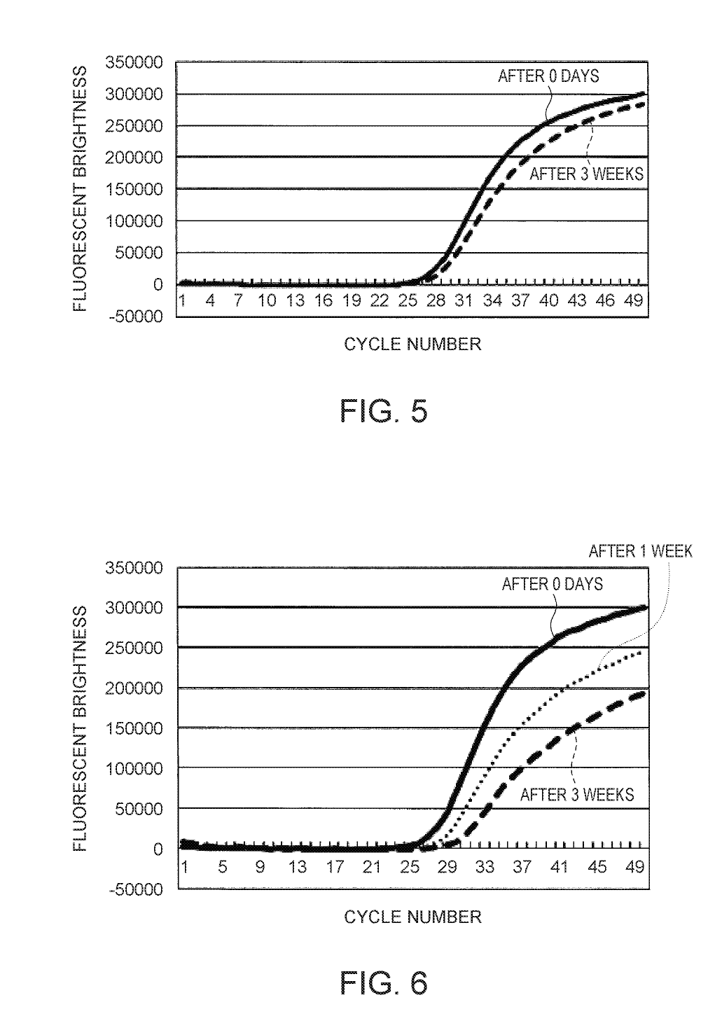

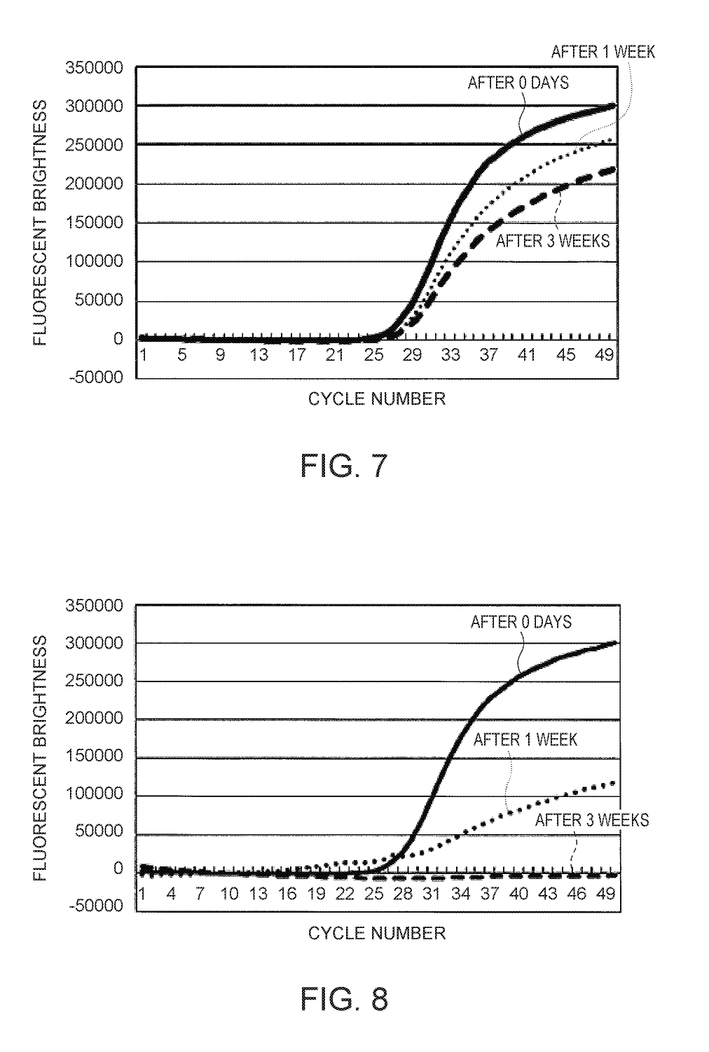

As shown in FIGS. 5 to 8, immediately after lyophilization, an amplification reaction was observed in the same manner in all cases. On the other hand, as shown in FIGS. 6 and 7, in the case where sucrose or raffinose was added in place of trehalose, a delay in the Ct value was observed after 1 week, however, an amplification reaction was confirmed also after 3 weeks. In this manner, in the case where an experiment was performed at the same concentration as in the case of trehalose, a storage stability effect was also obtained using sucrose and raffinose although the effect was not as much as in the case of using trehalose. As a result, it was found that even a trisaccharide can be used as the additive as long as it is a nonreducing sugar without limiting to a disaccharide.

Incidentally, as shown in FIG. 8, in the case where polypropylene which is used as an excipient of a lyophilized material was added, it was confirmed that the storage stability of the reagent was significantly deteriorated even after 1-week storage, and a storage stability effect as obtained in the other cases was not observed.

The invention is not limited to the above-mentioned embodiments, and various modifications can be made. For example, the invention includes substantially the same configurations (for example, configurations having the same function, method, and result, or configurations having the same object and effect) as the configurations described in the embodiments. Further, the invention includes configurations in which a non-essential portion in the configurations described in the embodiments is replaced. Further, the invention includes configurations having the same operational effects or the configurations capable of achieving the same objects as those of the configurations described in the embodiments. Further, the invention includes configurations in which a known technique is added to the configurations described in the embodiments.

The entire disclosure of Japanese Patent Application No. 2015-149483, filed Jul. 29, 2015 is expressly incorporated by reference herein.

What is claimed is:

1. A lyophilized reagent, for a nucleic acid amplification reaction obtained by lyophilizing a reaction mixture containing a reagent to be used for a nucleic acid amplification reaction and a sugar, wherein
the mass of the sugar is 72 mass % or more and 86 mass % or less with respect to the total mass of the solids contained in the lyophilized reagent.

2. The lyophilized reagent according to claim 1, wherein the sugar is a disaccharide or a trisaccharide.

3. The lyophilized reagent according to claim 1, wherein the lyophilized reagent is stored in a hydrophobic liquid.

4. The lyophilized reagent according to claim 3, wherein the hydrophobic liquid contains at least one member selected from a silicone oil, a mineral oil, and paraffin.

5. The lyophilized reagent according to claim 1, wherein the sugar contains trehalose.

6. The lyophilized reagent according to claim 1, wherein the reagent contains a reverse transcriptase.

7. The lyophilized reagent according to claim 1, wherein the reagent contains a polymerase and dNTP.

8. The lyophilized reagent according to claim 1, wherein the reagent contains at least one member selected from a primer and a probe.

9. The lyophilized reagent according to claim 1, wherein the sugar is contained in an amount of 77 mass % or more and 84 mass % or less with respect to the total mass of the solids contained in the lyophilized reagent.

10. A mixed reagent solution, for a nucleic acid amplification reaction including a reaction mixture containing a reagent to be used for a nucleic acid amplification reaction and a sugar, wherein
the concentration of the sugar in the mixed reagent solution is 10% (mass/volume) or more and 24% (mass/volume) or less.

11. A method for storing a lyophilized reagent, comprising storing the lyophilized reagent according to claim 1 in a hydrophobic liquid.

12. A method for storing a lyophilized reagent, comprising storing the lyophilized reagent according to claim 2 in a hydrophobic liquid.

13. A method for storing a lyophilized reagent, comprising storing the lyophilized reagent according to claim 3 in a hydrophobic liquid.

14. A method for storing a lyophilized reagent, comprising storing the lyophilized reagent according to claim 4 in a hydrophobic liquid.

15. A method for storing a lyophilized reagent, comprising storing the lyophilized reagent according to claim 5 in a hydrophobic liquid.

16. A method for storing a lyophilized reagent, comprising storing the lyophilized reagent according to claim 6 in a hydrophobic liquid.

17. A method for storing a lyophilized reagent, comprising storing the lyophilized reagent according to claim 7 in a hydrophobic liquid.

18. A method for storing a lyophilized reagent, comprising storing the lyophilized reagent according to claim 8 in a hydrophobic liquid.

19. A method for storing a lyophilized reagent, comprising storing the lyophilized reagent according to claim 9 in a hydrophobic liquid.
